(19) United States
(12) Patent Application Publication
(10) Pub. No.: US 2010/0184714 A1
(43) Pub. Date: Jul. 22, 2010

(54) Compositions For Delivering A Drug

(76) Inventors: Victor Albert Raul, Midland, MI (US); Linda Sue Nartker, Midland, MI (US)

Correspondence Address: HOWARD & HOWARD ATTORNEYS PLLC, 450 West Fourth Street, Royal Oak, MI, 48067, US

(21) Appl. No.: 12/668,492

(22) PCT Filed: Jul. 11, 2008
(86) PCT No.: PCT/US08/08545

§ 371 Date: Jan. 11, 2010

Related U.S. Application Data
(60) Provisional application No. 60/949,042, filed on Jul. 11, 2007.

(51) Int. Cl. A61K 31/7048; A61K 31/675; A61K 31/65; A61K 31/57; A61K 31/56; A61K 31/496; A61K 31/44; A61K 31/235; A61K 31/203; A61K 31/192; A61K 31/19; A61K 31/135; A61K 31/047; A61K 47/14

(52) U.S. Cl. 514/29; 514/91; 514/154; 514/177; 514/180; 514/253.08; 514/254.07; 514/356; 514/533; 514/559; 514/569; 514/573; 514/649; 514/729; 514/785

(57) ABSTRACT

A composition comprises a mixture of a silicone and a first solvent, a drug, such as a hydrophilic or lipophilic pharmaceutical agent, a salt of a dialkyl sulfosuccinate, such as dioctyl sodium sulfosuccinate (DOSS), and a second solvent, such as an alcohol. The salt of a dialkyl sulfosuccinate solubilizes the drug. The composition can be topically applied to a substrate, such as skin, for delivering the drug. Drugs with a wide solubility range are soluble and compatible in the composition without separation or crystallization of the drugs occurring.

### CROSS-REFERENCE TO RELATED APPLICATIONS

This application claims the benefit of U.S. Provisional Patent Application Ser. No. 60/949,042, which was filed on Jul. 11, 2007.

### FIELD OF THE INVENTION

The present invention relates to a composition for delivering a drug, and more specifically, to a composition comprising a mixture of a silicone and a first solvent, a drug, a salt of a dialkyl sulfosuccinate, and a second solvent different from the first solvent.

### DESCRIPTION OF THE RELATED ART

Compositions that include drugs, such as antibiotic salts, are well known in the healthcare art. These compositions are typically anhydrous and include petrolatum or polyethylene components, which are tactilely greasy. When a composition is greasy, patients are less likely to use or reuse the composition, thereby decreasing patient compliance. The anhydrous compositions are typically silicone based and lipophilic, which poses formulation problems when trying to use hydrophilic drugs. For example, an antibiotic salt such as clindamycin phosphate is known to be hydrophilic, e.g. the antibiotic salt is soluble in water, hence one would not expect to formulate clindamycin phosphate in a lipophilic anhydrous composition. While some hydrophilic drugs have been formulated in anhydrous compositions, crystals, polymorphs, and agglomerations of the crystals can form over time. Uncontrolled crystal formation and agglomeration in the anhydrous compositions yields crystals having an average diameter of greater than 10 microns, typically ranging from 15 to 20 microns or higher. Such uncontrolled crystal formation and agglomerations thereof result in physical instability of the anhydrous compositions, decrease in bioavailability and/or bioequivalence.

Anhydrous compositions including tetracycline antibiotic salts are disclosed, for example, in U.S. Pat. No. 4,376,118 to Daher at al. (the '118 patent). The anhydrous compositions of the '118 patent include a mixture of a tetracycline antibiotic salt, a nonaqueous diluent, a nonaqueous solvent and a nonaqueous solubilizer. While the anhydrous compositions of the '118 patent are stable over time, the '118 patent makes clear that the preferred nonaqueous nonionic solubilizer is a polyethylene glycol (PEG). For example, the examples of the '118 patent disclose use of polyethylene glycol-200 as a nonaqueous nonionic solubilizer for making the anhydrous compositions. As previously described above, polyethylene components such as the PEG of the '118 patent, are greasy which decreases patient compliance. In addition, PEG can form peroxides, which can make drugs other than a tetracycline antibiotic salt unstable and can also inhibit delivery of the drug into skin.

Accordingly, there remains an opportunity to provide an improved composition that provides desirable aesthetic properties, such as non-greasiness after application to skin, which is a key to patient compliance.

### SUMMARY OF THE INVENTION AND ADVANTAGES

The present invention provides a composition comprising a mixture of a silicone and a first solvent, a drug, a salt of a dialkyl sulfosuccinate, and a second solvent different from the first solvent. The salt of a dialkyl sulfosuccinate solubilizes the drug. The present invention further provides a method of delivering a drug to a substrate.

The present invention provides a unique combination of the mixture of the silicone and the first solvent, the drug, the salt of a dialkyl sulfosuccinate, and the second solvent to make the composition. Drugs with a wide solubility range are soluble and compatible in the compositions of the present invention. Amounts of the salt of a dialkyl sulfosuccinate and the second solvent can be adjusted to maintain near saturation of the drug for optimum delivery and release of the drug into substrates such as skin, and therapeutic effectiveness of the drug does not change over time. The composition does not have a deleterious medium that can cause instability of the drug. In addition, water, pH adjustment, and heat are generally not required for preparing the composition. The composition can also have desirable aesthetic properties, specifically, tactile feel, such as non-greasiness after application to skin.

### DETAILED DESCRIPTION OF THE INVENTION

The composition of this invention may be used for various applications known to those skilled in the healthcare art. Examples of these applications include, but are not limited to, epidermal, dermal, and hypodermal skin applications, pharmaceutical, antimicrobial, and nutrient applications, internal applications such as oral, nasal, and implantable applications, and veterinary applications. The composition is especially suited for use in liquid form as a topical composition, e.g. as a liquid lotion, for use on skin, wherein one or more of the components, such as a drug, can permeate into one or more layers of the skin. The composition may be used for treating various ailments such as acne or psoriasis. The composition can also be used in semi-solid or solid forms such as for transdermal patches and implantable devices.

The composition is typically anhydrous. By “anhydrous”, it is meant that the composition is substantially free of water. However, it is to be appreciated that the composition may have some water content due to residual water in one or more of the components of the composition, and/or due to atmospheric moisture absorbed by the composition. If water is present in the composition, the composition typically includes water in an amount less than 5, alternatively less than 1, alternatively approaching or equaling 0, parts by weight, based on 100 parts by weight of the composition. Water is normally undesirable in the composition because water can cause the composition to become unstable and separate over time depending in part on the specific components, and the amounts thereof, employed to make the composition. Water can also cause instability or degradation of certain components, such as a drug, which is described further below, and may cause uncontrolled side reactions of the components employed to make the composition.

The composition comprises a mixture of a silicone and a first solvent, a drug, a salt of a dialkyl sulfosuccinate, and a second solvent different from the first solvent. The silicone may be selected from the group of a silicone elastomer, a silicone adhesive, e.g. a silicone pressure sensitive adhesive (PSA), a liquid silicone rubber, a hydroxyl-terminated polydimethylsiloxane gum, and combinations thereof.

Silicone elastomers useful herein typically comprise the reaction product of an containing polysiloxane and an alpha,omega-diene. The ≡Si—H containing polysiloxane can have the formula

R₃SiO(R′₂SiO)ₐ(R″HSiO)_{b}SiR₃ (I)

wherein R, R′, and R″ are typically each independently alkyl groups with 1 to 6 carbon atoms, a is 0 to 250, and b is 1 to 250. Other suitable Si—H containing polysiloxanes for making the silicone elastomer include those having the formula

HR₂SiO(R′₂SiO)ₙSiR₂H (II)

and/or the formula

HR₂SiO(R′₂SiO)ₐ(R″HSiO)_{b}SiR₂H (III)

wherein R, R′, and R″ are typically each independently alkyl groups with 1 to 6 carbon atoms, a is 0 to 250, b is 1 to 250, and c is 0 to 250. Typically, the Si—H containing polysiloxane comprises polysiloxane (I) and at least one of polysiloxane (II) or (III). The alpha,omega-diene is typically of the formula

CH₂═CH(CH₂)ₓCH═CH₂

wherein x is typically 1 to 20. Suitable alpha,omega-dienes for making the silicone elastomer include, but are not limited to, 1,4-pentadiene, 1,5-hexadiene, 1,6-heptadiene, 1,7-octadiene, 1,8-nonadiene, 1,9-decadiene, 1,11-dodecadiene, 1,13-tetradecadiene; and 1,19-eicosadiene, and combinations thereof.

The silicone elastomer is typically made in the presence of a metal catalyst for reacting and cross-linking the Si—H containing polysiloxane and the alpha,omega-diene. Suitable metal catalysts for making the silicone elastomer include Group VIII transition metal catalysts, such as a platinum catalyst. Other suitable metal catalysts for making the silicone elastomer include, but are not limited to, tin catalysts.

When the silicone is a silicone elastomer, the first solvent typically comprises a polysiloxane. The polysiloxane is useful for swelling the silicone elastomer. Specifically, when the first solvent is combined with the silicone elastomer to form the mixture, the first solvent serves to suspend and swell the silicone elastomer to provide an elastic three-dimensional network or matrix, e.g. a gel, which can quickly be formed by applying shear force to the mixture, such as by using a dispersion blade, a dental mixer, etc., to blend the silicone elastomer and the first solvent to form the mixture.

In the mixture of the silicone elastomer and the polysiloxane, the polysiloxane is typically a low molecular weight linear polysiloxane, a low molecular weight cyclic polysiloxane, or a mixture of low molecular weight linear and cyclic polysiloxanes. Suitable polysiloxanes for making the composition are typically selected from the group consisting of, but are not limited to, hexamethyldisiloxane, hexamethylcyclotrisiloxane, hexadecamethylheptasiloxane, octamethyltrisiloxane, octamethylcyclotetrasiloxane, decamethyltetrasiloxane, decamethylcyclopentasiloxane, dodecamethylpentasiloxane, dodecamethylcyclohexasiloxane, tetradecamethylhexasiloxane, and combinations thereof. Typically, the polysiloxane is a cyclosiloxane, more typically decamethylcyclopentasiloxane, which is commonly referred to in the art as “D5”. Other suitable polysiloxanes for making the mixture of the silicone elastomer and the first solvent include low molecular weight linear alkyl polysiloxanes, low molecular weight aryl polysiloxanes, or mixtures thereof. If employed, the low molecular weight linear polysiloxanes are typically of the formula

R₃SiO(R₂SiO)_{y}SiR₃

wherein R is typically selected from the group of alkyl groups containing 1 to 6 carbon atoms and/or aryl groups such as phenyl, and y is typically a value to impart the first solvent with a viscosity generally less than 100 mm²/s.

The first solvent, e.g. polysiloxane, is typically a liquid under ambient conditions and typically has a low viscosity for improved application and spreading of the composition onto skin. The first solvent, e.g. D5, is typically volatile, to at least partially evaporate after the composition is applied to skin; however, the first solvent can be non-volatile, depending on the specific first solvent employed to make the composition. The term “volatile”, as used herein, means that the first solvent has a vapor pressure of more than 0.2 mm Hg at 25° C. at one atmosphere and/or has a boiling point of less than 300° C. at one atmosphere. It is to be appreciated that the first solvent can be present during manufacture of the silicone elastomer. In other words, the first solvent can be inherent in the mixture with the silicone elastomer, and/or the first solvent can be added after the silicone elastomer is made.

The mixture may be made with the containing polysiloxane and the alpha,omega-diene to form a gel by crosslinking and addition of SiH across double bonds in the alpha,omega-diene, typically with a nonvolatile content of 8% to 18%, alternatively from 12% to 13%, in a cyclomethicone, e.g. D5. In this embodiment, the mixture is generally assigned the INCI name: “cyclomethicone (and) dimethicone crosspolymer”. In another embodiment, the mixture comprises a “dimethicone (and) dimethicone crosspolymer”. If employed, the crosspolymer is typically a loosely cross-linked crosspolymer. It is believed that the loose cross-linking of the crosspolymer is useful for imparting the composition with desirable viscosity and aesthetic properties, as further described below.

Specific suitable examples of the mixture of the silicone elastomer and the first solvent are Dow Corning® ST-Elastomer 10, Dow Corning® 9040 Silicone Elastomer Blend, and Dow Corning® 9041 Silicone Elastomer Blend, all of which are commercially available from Dow Corning Corporation of Midland, Mich. Further suitable mixtures of the silicone elastomer and the first solvent are disclosed in U.S. Pat. Nos. 5,654,362 and 5,888,210, both to Schultz et al., which are incorporated herein by reference in their entirety. The silicone elastomer is typically present in an amount of 5 to 20, alternatively from 10 to 15, alternatively from 12 to 13, parts by weight, based on 100 parts by weight of the mixture. If the first solvent is the polysiloxane, the first solvent is typically present in an amount of 45 to 90, alternatively from 55 to 90, alternatively from 60 to 88, parts by weight, based on 100 parts by weight of the mixture.

The silicone elastomer and the first solvent are typically present in a weight ratio of 5:1 to 9:1. This mixture has a viscosity that is useful for imparting the composition with ease of application. The mixture is typically present in an amount of 60 to 98 parts by weight, based on 100 parts by weight of the composition. However, for purposes of the present invention, it is to be appreciated that various amounts of the mixtures may be used, other than those amounts described herein, such as quantities sufficient (qs) to achieve a desirable property such as viscosity and/or dosing of the drug.

The silicone may also be a silicone adhesive, typically a silicone based pressure sensitive adhesive (PSA), which can be used in transdermal reservoir patches or devices. Typically, the silicone adhesive comprises the reaction product of a polydialkyldimethylsiloxane end blocked with silanols and a silicate resin. In certain embodiments, the silicone adhesive comprises the condensation reaction product of a polydimethylsiloxane end blocked with silanols and a silicate resin. Specific examples of suitable silicone adhesives are Dow Corning® 7-9800 Soft Skin Adhesive, Dow Corning® BIO-PSA 7-4502 Silicone Adhesive, Dow Corning® BIO-PSA 7-4602 Silicone Adhesive, Dow Corning® 7-4101, Dow Corning® 7-4102, Dow Corning® 7-4201, Dow Corning® 7-4202, Dow Corning® 7-4301, Dow Corning® 7-4302, Dow Corning® 7-4401, Dow Corning® 7-402, Dow Corning® 7-4501, Dow Corning® 7-4601, and Dow Corning® 7-4560, all of which are commercially available from Dow Corning Corporation.

When the silicone is the silicone adhesive, the first solvent is typically selected from the group of alkanes, such as heptane; arenes, such as toluene; and esters, such as ethyl acetate; and combinations thereof. In this embodiment, the first solvent serves as a processing aid, which is further described below. It is to be appreciated that the silicone adhesive may also be applied to other substrates known in the art, such as patches. In certain embodiments, when the silicone adhesive is employed as the mixture, the composition is substantially free of or completely free of the second solvent. In these embodiments, the second solvent can also serve as a processing aid, if employed. The silicone adhesive is typically present in at least 50 parts by weight based on 100 parts of the silicone adhesive and the first solvent combined, i.e., the mixture. The silicone adhesive is typically present in an amount of 40 to 80, alternatively from 50 to 70, alternatively from 60 to 65, parts by weight, based on 100 parts by weight of the mixture. The first solvent, e.g. ethyl acetate, is typically present in an amount of 1 to 40, alternatively from 25 to 40, alternatively from 25 to 30, parts by weight, based on 100 parts by weight of the mixture.

The silicone may also be a dimethiconol, which is a dimethyl siloxane terminated with hydroxyl groups. Specific examples of suitable dimethiconols are Dow Corning® Dimethiconal Blend 20 and ST-Dimethiconol 40, both of which are commercially available from Dow Corning Corporation. Other suitable solvents, for purposes of the present invention, include Dow Corning® 200 Fluid, e.g. Dow Corning® 200 Fluid, 5 CST, Dow Corning® 245 Fluid, isododecane, isohexadecane, and isodecylneopentanoate.

The silicone may also be a liquid silicone rubber. When the silicone is the liquid rubber silicone, the silicone is typically present in an amount of 75 to 95 parts by weight and the first solvent is typically present in an amount of 1 to 50 parts by weight, each based on 100 parts by weight of the composition.

Some of the mixtures described above and other mixtures of the silicone and the first solvent suitable for making the composition are disclosed by various patents and publications including U.S. Pat. Nos. 4,882,377 to Sweet et al., 4,987,169 to Kuwata et al., 5,599,533 to Stepniewski et al., 5,654,362 to Schulz Jr. et al., 5,811,487 to Schulz Jr. et al., 5,880,210 to Schulz Jr. et al., 5,889,108 to Zhang, 5,929,164 to Zhang, 5,948,855 to Lin et al., 5,969,035 to Meinhardt et al., 5,977,280 to Kadlec et al., 5,994,459 to Berg et al., 6,015,858 to Gornowicz, 6,027,738 to Stepniewski et al., 6,080,394 to Lin et al., 6,168,782 to Lin et al., 6,177,071 to Lin et al., 6,200,581 to Lin et al., 6,207,717 to Lin et al., 6,221,927 to Lin et al., 6,221,979 to Lin et al., 6,238,657 to Lin et al., 6,346,583 to Kilgour et al., 6,444,745 to Kilgour et al., 6,538,061 to Chaiyawat et al., and U.S. Patent Application Publication No. 2004/0228821 to Sunkel et al., the disclosures of which are incorporated herein by reference in their entirety. It is to be appreciated that the mixture of the present invention may comprise any combination of one or more of the silicones and one or more of the first solvents described and exemplified above.

The mixture of the silicone elastomer and the first solvent is typically in the form of the gel, which imparts the composition with desirable viscosity and aesthetic properties, improves stability of the composition, and suspends and carries the drug within the composition, which is further described below. When the composition has desirable aesthetic properties, specifically, tactile feel, it is believed that consumers are more likely to use or comply with use of the composition due to increased sensory satisfaction and ease of application. This increases overall patient compliance. For example, after the composition is topically applied to the consumer's skin and at least partially absorbed and/or evaporated, some representative aesthetic properties of the post-applied composition include film residue, greasiness, silkiness, tackiness, slipperiness, and gloss. If the composition is compared to another composition known in the art, such as petrolatum, e.g. petroleum jelly, the composition of the present invention generally has lower film residue, lower greasiness, lower tackiness, and lower gloss, and generally has higher silkiness and higher slipperiness, relative to the petrolatum. These aesthetic properties of the composition are typically more desired by the patient relative to the properties of the petrolatum. While the gel is generally employed, the mixture of the silicone elastomer and the first solvent can also be in other forms known to those of ordinary skill in the healthcare art. For example, and as alluded to above, the mixture can be a liquid, a semi-solid, a solid, a blend, an emulsion, or a paste, which imparts the composition with the corresponding viscosity and aesthetic properties of the mixture.

The drug may be any drug known in the healthcare art, and typically, is a pharmaceutical agent such as a pharmaceutical or antibiotic salt, a vitamin derivative, or an antimicrobial agent. The composition can also include any combination of two of more different drugs. The composition is especially useful for carrying various drugs with a wide solubility range, such as pharmaceutical agents that are hydrophilic or lipophilic. Examples of suitable drugs for making the composition can be selected from the group of, but are not limited to, tetracycline, clindamycin phosphate, erythromycin, ciprofloxacin, norfloxacin, salicylic acid, benzoyl peroxide, alprostadil, terbinafine, clobetasol propionate, ketoconazole, progesterone, niacinamide, calcipotriol, retinoids (including retinoic acid, cis-retinoic acid/isotretinoin, trans-retinoic acid/tretinoin, tazarotene, and adapalene), vitamin D analogues, vitamin A esters, dapsone, doxycycline, and combinations thereof. The drug is typically present in an amount of 0.001 to 15 parts by weight, based on 100 parts by weight of the composition.

The salt of a dialkyl sulfosuccinate is typically dioctyl sodium sulfosuccinate (DOSS), which is also referred to in the art as docusate sodium or sodium docusate. The salt of a dialkyl sulfosuccinate solubilizes the drug, and is especially useful for solubilizing hydrophilic and lipophilic pharmaceutical agents and regulating the viscosity of the composition. Specifically, in certain embodiments, the drugs are completely solubilized and typically remain stable in the composition without agglomerating or crystallizing within the composition. Large crystal formation, such as crystals having average diameters of greater than 10 microns, and agglomerations of the crystals of the drug are undesirable as they tend to hinder the drug's effectiveness and can also be aesthetically displeasing both visually and tactilely to a consumer of the composition. It is believed that using DOSS can also have a synergistic effect when antimicrobials are employed as the drug, due to stabilization effects. Typically, since the salt of a dialkyl sulfosuccinate, in combination with a low molecular weight alcohol, e.g. ethanol (described below), solubilizes the drug, no heat or pH adjustment is needed in order to make the composition, e.g. to incorporate the drug within the composition. As understood in the art, heat and pH can cause some drugs to loss effectiveness by degradation. Many drug degradation products are toxic, which are undesirable. The salt of a dialkyl sulfosuccinate is typically present in an amount of 0.01 to 5 parts by weight, based on 100 parts by weight of the composition.

In certain embodiments, depending in part on the amount of the drug employed in the composition, some portion of the drug will not be completely solubilized. In other words, in these embodiments, some amount of the drug will be solubilized and some amount of the drug will be in a disperse phase, typically in the form of small crystals having an average diameter of no greater than 10 microns, more typically having an average diameter from about 5 to about 10 microns. In these embodiments, crystal formation is controlled, such that agglomeration and therefore settling of the drug crystals is slowed, minimized, or nonexistent, due in part to the viscosity of the composition. Further, the composition is typically at steady-state, such that when the composition is employed by a consumer, the drug that is solubilized is absorbed into a consumer's skin, and the drug that is absorbed is replaced by drug that is then solubilized from the disperse phase. In other words, the crystals, if present, are solubilized from the disperse phase replace the pre-solubilized drug that is adsorbed and/or permeates into the consumer's skin, such that a near constant rate of drug diffusion of the drug is achieved until the drug is absorbed into the consumer's skin from the composition.

The second solvent is typically an alcohol, more typically a monomeric alcohol, such as an alcohol having from 1 to 10 carbon atoms and one hydroxyl group. Generally, for purposes of the present invention, alcohols having greater than 10 carbon atoms have undesirable melting points, which can lead to solidifaction of the composition (or portions thereof). As such, the alcohol may be selected from the group of methanol, ethanol, propanol, butanol, pentanol, hexanol, heptanol, octanol, nonanol, decanol, and combinations thereof. It is to be appreciated that the alcohol may also be various isomers of the alcohols described above, such as, but not limited to, 1-propanol, 1-butanol, and 1-pentanol. Typically, the alcohol is ethanol. The second solvent solubilizes the salt of a dialkyl sulfosuccinate and can also solubilize the drug. The second solvent is typically substantially free from esters, which can hydrolyze and form water, which is undesired for the reasons described and exemplified above. The salt of a dialkyl sulfosuccinate and the alcohol are typically present in a weight ratio of 1:1 to 1:4. Combining the salt of a dialkyl sulfosuccinate and the alcohol is useful for solubilizing the salt of a dialkyl sulfosuccinate, and therefore the drug, in the composition. For example, pharmaceutical salts typically crystallize in lipophilic matrices such as the gel. By controlling the amount of the salt of a dialkyl sulfosuccinate and the second solvent, the drug can be added to the composition in higher levels, such as being present near saturation levels. Further, as alluded to above, in certain embodiments, the drug can be present in the disperse phase, such as present in levels over a saturation level. Specifically, the second solvent, in combination with the salt of a dialkyl sulfosuccinate, is especially useful for dispersing, suspending and/or solubilizing the drug in the form of encapsulated drugs, or vesicles, or other thermodynamically stable associative phases or structures. The second solvent is typically present in an amount of 0.1 to 20, alternatively from 0.1 to 10, parts by weight, based on 100 parts by weight of the composition.

The composition may further comprise an additive known to those of ordinary skill in the healthcare art. For example, the additive can be selected from the group of an emollient; a penetration enhancer; a fragrance; a drug solubilizer; an antioxidant such as butylhydroxytoluene (BHT); UV light blockers such as titanium dioxide (TiO₂); steroids such as betamethasone; pH adjusters such as acids or bases, e.g. sodium bicarbonate or sodium citrate; and combinations thereof.

If employed, the emollient may be any emollient known in the art. The emollient is useful for softening and soothing skin of a consumer of the composition. The emollient is typically selected from the group of diisopropyl adipate, diisopropyl sebacate, triethyl citrate, isopropyl myristate, isopropyl palmitate, myristyl propionate, 2-ethylhexyl palmitate, cetyl palmitate, cetyl stearate, triglycerides, fatty acids, fatty alcohols (e.g. an oleyl alcohol), and combinations thereof. A suitable triglyceride is caprylicicapric triglyceride and a suitable fatty acid is caprylic acid. If employed, the emollient is typically present in an amount of 0.1 to 50, alternatively from 0.1 to 25, alternatively from 0.1 to 10, alternatively from 0.1 to 5, parts by weight, based on 100 parts by weight of the composition. In addition to solubilizing the drug into the mixture, it is believed that the salt of a dialkyl sulfosuccinate, e.g. DOSS, is useful for imparting compatibility between the mixture and the emollient, e.g. oleyl alcohol, if employed. For example, if one were to mix oleyl alcohol with just the mixture, the two would separate. However, with the addition of a suitable amount of the salt of a dialkyl sulfosuccinate, separation between the mixture and the emollient generally does not occur. Such an amount of the salt of a dialkyl sulfosuccinate depends on amounts of each of the components, and can be determined via routine experimentation.

If employed, the penetration enhancer may be any penetration enhancer known in the art. The penetration enhancer is useful for facilitating penetration of the drug into the skin of a consumer of the composition. The penetration enhancer is typically selected from the group of oleic acid, linoleic acid, glycerin monolaurate, azone (1-dodecylazacycloheptan-2-one), dodecyl 2-(N,N-dimethylamino)propionate, dodecyl 2-(N,N-dimethylamino)propionate.HCl, propylene glycol monolaurate, propylene glycol, dipropylene glycol, and combinations thereof. If employed, the penetration enhancer is typically present in an amount of 0.01 to 10, alternatively from 0.01 to 5, alternatively from 0.5 to 2, parts by weight, based on 100 parts by weight of the composition.

If employed, the surfactant may be any surfactant known in the art. Depending on conditions, the surfactant can also be referred to in the art as an emulsifier. The surfactant is useful for imparting homogeneity to the composition, drug solubilization, and based on its partition coefficient, the surfactant can be a penetration enhancer for permeation into skin. Examples of suitable surfactants, for purposes of the present invention, are generally nonionic surfactants, and include polysorbates, such as polysorbate 80, and sorbitan esters, such as sorbitan monolaurate, sorbitan monoleate, and sorbitan sesquioleate. If employed, the surfactant is typically present in an amount of 0.01 to 10, alternatively from 0.01 to 5, alternatively from 0.5 to 2, parts by weight, based on 100 parts by weight of the composition.

The composition may be prepared by combining the silicone, the first solvent, the drug, the salt of a dialkyl sulfosuccinate, and the second solvent. In one embodiment, the salt of a dialkyl sulfosuccinate is solubilized in the second solvent to form a blend. The drug is then mixed with the blend and the mixture of the silicone and the first solvent, e.g. cyclomethicone and dimethicone crosspolymer, is then added as a viscosity builder and matrix to make the composition. In another embodiment, the salt of a dialkyl sulfosuccinate is melted to form a melt. The drug is then mixed into the melt. The second solvent and the mixture of the silicone and the first solvent are then added to make the composition. Optionally, the additive such as the emollient and/or the penetration enhancer is added to the composition. The composition can be made in a vessel using various mixing and dispersion blades. Typically, a pH change is not necessary to prepare the composition. In addition, application of heat is optional, depending on the salt of a dialkyl sulfosuccinate employed, and if heat is applied to the salt of a dialkyl sulfosuccinate, heat is not necessary for the composition as a whole.

If the silicone adhesive, e.g. PSA, is employed, the salt of a dialkyl sulfosuccinate is solubilized in the second solvent to form a blend. The drug is then mixed with the silicone adhesive (including the first solvent) to form the composition. The composition can then be coated on a release liner and the first solvent is allowed to volatize off via heat or ambient conditions, which results in a film, solid matrix, or patch, which can be applied to skin to provide a systemic or local therapeutic effect via the drug.

As alluded to above, the composition prepared herein can be applied to a substrate to deliver the drug. Upon application of the composition, a film is typically formed on the substrate. The film contains the drug, which is delivered from or through the film to the substrate. If the substrate is skin, the composition is applied to the skin to deliver the drug to the skin. The composition may be applied in various ways, such as by rubbing or coating the composition directly onto the skin. Alternatively, as described above, the composition may be applied on a transdermal patch prior to application of the transdermal patch to the skin.

The following examples, illustrating the compositions of the present invention, are intended to illustrate and not to limit the present invention.

### EXAMPLES

Examples 1 through 36 of the composition of the present invention are prepared. The compositions are made by combining the mixture of the silicone and the first solvent, the drug, the salt of a dialkyl sulfosuccinate, the second solvent, and optionally, one of more of the additives, e.g. the emollient and the penetration enhancer, using standard blending methods. Generally, the drug is first dispersed in the salt of a dialkyl sulfosuccinate and the second solvent to form a blend and the blend is then added to the mixture to form the composition. Depending on the solvents employed, the solvents may or may not volatilize from the compositions. Commonly used stainless steel vessels are used for manufacturing the compositions. A closed vessel or tank with nitrogen purging/sparging may be necessary if the drug is susceptible to oxidation, or photo-oxidation. Various mixing means may be employed, such as propeller mixers or dental mixers. The compositions are allowed to sit undisturbed to visually determine if any settling or separation occurs. The compositions are also inspected under microscope, specifically under a 100× magnification, to determine if any agglomerations or crystals form within the compositions, especially with regard to the drug. The amount and type of each component used to form the composition is indicated in Tables 1 through 8 below with all values in parts by weight based on 100 parts by weight of the composition unless otherwise indicated.

TABLE 1

| | Example | | | | | | | |
|---|---|---|---|---|---|---|---|---|
| | 1 | 2 | 3 | 4 | 5 | 6 | 7 | 8 |
| Mixture 1 | 94.06 | 94.06 | 94.56 | 89.95 | 92.95 | 96.95 | 83.00 | 90.50 |
| Drug 1 | 0.44 | 0.44 | 0.44 | — | — | — | — | — |
| Drug 2 | — | — | — | 0.05 | 0.05 | 0.05 | — | — |
| Drug 3 | — | — | — | — | — | — | 2.00 | — |
| Drug 4 | — | — | — | — | — | — | — | 1.00 |
| Salt of a Dialkyl | 2.00 | 2.00 | 2.00 | 2.00 | 2.00 | 1.00 | 2.00 | 1.50 |
| Sulfosuccinate | | | | | | | | |
| Second Solvent 1 | 3.00 | 3.00 | 3.00 | 3.00 | 2.00 | 2.00 | 3.00 | 2.00 |
| Emollient | — | — | — | 5.00 | 5.00 | — | 10.00 | 5.00 |
| Penetration | 0.50 | — | — | — | — | — | — | — |
| Enhancer 1 | | | | | | | | |
| Penetration | — | 0.50 | — | — | — | — | — | — |
| Enhancer 2 | | | | | | | | |
| Total | 100.00 | 100.00 | 100.00 | 100.00 | 102.00 | 100.00 | 100.00 | 100.00 |

Mixture 1 is a high molecular weight silicone elastomer in decamethylcyclopentasiloxane (D5) in the form of a gel, commercially available from Dow Corning Corporation of Midland, Mich. under the trade name of Dow Corning® ST-Elastomer 10. The high molecular weight silicone elastomer is present in an amount of 12 to 13 parts by weight, and the D5 is present in an amount of 87 to 88 parts by weight, each based on 100 parts by weight of the mixture.

Drug 1 is alprostadil.

Drug 2 is clobetasol propionate.

Drug 3 is clindamycin phosphate.

Drug 4 is dapsone.

Salt of a Dialkyl Sulfosuccinate is dioctyl sodium sulfosuccinate (DOSS), commercially available from Spectrum Chemical Mfg. Corp. of Gardena, Calif.

Second Solvent 1 is ethanol.

Emollient is diisopropyl sebacate, commercially available from Croda Inc. of Edison, N.J.

Penetration Enhancer 1 is dodecyl 2-(N,N-dimethylamino)propionate (DDAIP), commercially available from NexMed Inc. of East Windsor, N.J.

Penetration Enhancer 2 is dodecyl 2-(N,N-dimethylamino)propionate-HCl (DDAIP HCl), commercially available from NexMed Inc.

TABLE 2

| | Example | | | | | | | |
|---|---|---|---|---|---|---|---|---|
| | 9 | 10 | 11 | 12 | 13 | 14 | 15 | 16 |
| Mixture 1 | 94.00 | 87.00 | 54.50 | 65.00 | 90.00 | 88.00 | 89.00 | 93.00 |
| Drug 3 | 2.00 | — | — | — | — | — | — | 2.00 |
| Drug 4 | — | 1.00 | — | — | — | — | — | — |
| Drug 5 | — | — | 10.00 | 10.00 | — | — | — | — |
| Drug 6 | — | — | — | — | 1.00 | — | — | — |
| Drug 7 | — | — | — | — | — | 2.00 | — | — |
| Drug 8 | — | — | — | — | — | — | 2.00 | — |
| Salt of a Dialkyl | 2.00 | 2.00 | 5.00 | 5.00 | 1.00 | 2.00 | 2.00 | 2.00 |
| Sulfosuccinate | | | | | | | | |
| Second Solvent 1 | 2.00 | 2.00 | 20.00 | 10.00 | 2.00 | 3.00 | 3.00 | 3.00 |
| Emollient | — | 10.00 | 10.00 | 10.00 | 6.00 | 5.00 | 4.00 | — |
| Penetration | — | — | 0.50 | — | — | — | — | — |
| Enhancer 1 | | | | | | | | |
| Total | 100.00 | 102.00 | 100.00 | 100.00 | 100.00 | 100.00 | 100.00 | 100.00 |

Drug 5 is benzoyl peroxide.

Drug 6 is ciprofloxacin.

Drug 7 is erythromycin.

Drug 8 is doxycycline.

TABLE 3

| | Example | | | | | | |
|---|---|---|---|---|---|---|---|
| | 17 | 18 | 19 | 20 | 21 | 22 | 23 |
| Mixture 1 | 96.50 | 97.40 | 97.50 | — | — | — | — |
| Mixture 2 | — | — | — | 93.00 | 85.00 | 91.00 | 85.00 |
| Drug 1 | — | — | — | — | — | 4.00 | — |
| Drug 3 | — | — | — | 2.00 | 5.00 | — | — |
| Drug 4 | — | — | — | — | — | — | 5.00 |
| Drug 9 | 0.0003 | — | — | — | — | — | — |
| Drug 10 | — | 0.100 | 0.500 | — | — | — | — |
| Salt of a Dialkyl | 1.00 | 0.80 | 0.40 | 2.00 | 4.00 | 2.00 | 4.00 |
| Sulfosuccinate | | | | | | | |
| Second Solvent 1 | 2.50 | 1.20 | 0.60 | 3.00 | 6.00 | 3.00 | 6.00 |
| Penetration | — | 0.50 | 1.00 | — | — | — | — |
| Enhancer 3 | | | | | | | |
| Total | 100.00 | 100.00 | 100.00 | 100.00 | 100.00 | 100.00 | 100.00 |

Mixture 2 is a high molecular weight silicone elastomer in D5, commercially available from Dow Corning Corporation under the trade name of Dow Corning® 9040 Silicone Elastomer Blend. The high molecular weight silicone elastomer is present in an amount of 12 to 13 parts by weight, and the D5 is present in an amount of 87 to 88 parts by weight, each based on 100 parts by weight of the mixture.

Drug 9 is calcipotriol.

Drug 10 is retinoic acid.

Penetration Enhancer 3 is oleic acid.

TABLE 4

| | Example | | | | |
|---|---|---|---|---|---|
| | 24 | 25 | 26 | 27 | 28 |
| Mixture 3 | 90.00 | 96.00 | — | — | — |
| Mixture 4 | — | — | 90.00 | 85.00 | — |
| Mixture 5 | — | — | — | — | 88.00 |
| Drug 1 | — | 1.00 | — | — | — |
| Drug 2 | 5.00 | — | — | — | — |
| Drug 4 | — | — | — | 5.00 | — |
| Drug 11 | — | — | 5.00 | — | — |
| Drug 12 | — | — | — | — | 7.00 |
| Salt of a | 2.00 | 1.00 | 2.00 | 4.00 | 2.00 |
| Dialkyl | | | | | |
| Sulfo- | | | | | |
| succinate | | | | | |
| Second | 3.00 | 2.00 | 3.00 | 6.00 | 3.00 |
| Solvent 1 | | | | | |
| Total | 100.00 | 100.00 | 100.00 | 100.00 | 100.00 |

Mixture 3 is a two part (parts A and B) silicone adhesive, which is a platinum-catalyzed polydimethylsiloxane composition, in a 1 part A to 1 part B ratio, commercially available from Dow Corning Corporation under the trade name of Dow Corning® 7-9800 Soft Skin Adhesive.

Mixture 4 is a two part (parts A and B) silicone, which is tin-catalyzed, in a 9 parts A to 1 part B ratio, commercially available from Dow Corning Corporation.

Mixture 5 is a two part (parts A and B) liquid silicone rubber, which is a platinum-catalyzed cross-linked dimethyl and methyl-vinyl siloxane copolymer composition having reinforcing silica, in a 1 part A to 1 part B ratio, commercially available from Dow Corning Corporation.

Drug 11 is ketoconazole.

Drug 12 is progesterone.

TABLE 5

Example

| | 29 | 30 | 31 | 32 |
|---|---|---|---|---|
| Mixture 5 | 92.00 | — | — | — |
| Mixture 6 | — | 90.00 | — | — |
| Mixture 7 | — | — | 80.00 | 96.50 |
| Drug 10 | — | — | — | 0.500 |
| Drug 12 | 3.00 | — | — | — |
| Drug 13 | — | 5.00 | 10.00 | — |
| Salt of a Dialkyl | 2.00 | 2.00 | 4.00 | 0.80 |
| Sulfosuccinate | | | | |
| Second Solvent 1 | 3.00 | 3.00 | — | 1.20 |
| Second Solvent 2 | — | — | 6.00 | — |
| Penetration | — | — | — | 1.00 |
| Enhancer 3 | | | | |
| Total | 100.00 | 100.00 | 100.00 | 100.00 |

Mixture 6 is a silicone adhesive in ethyl acetate, commercially available from Dow Corning Corporation under the trade name of BIO-PSA® 7-4602 Silicone Adhesive. The silicone adhesive is present in an amount of 60 parts by weight, and the ethyl acetate is present in an amount of 40 parts by weight, each based on 100 parts by weight of the mixture.

Mixture 7 is a silicone adhesive in ethyl acetate, commercially available from Dow Corning Corporation under the trade name of BIO-PSA® 7-4502 Silicone Adhesive. The silicone adhesive is present in an amount of 65 parts by weight, and the ethyl acetate is present in an amount of 35 parts by weight, each based on 100 parts by weight of the mixture.

Drug 13 is niacinamide.

Second Solvent 2 is isopropanol.

TABLE 6

| | Example | | | | |
|---|---|---|---|---|---|
| | 33 | 34 | 35 | 36 | 37 |
| Mixture 1 | 99.50 | 96.40 | 97.40 | 94.70 | 92.00 |
| Drug 5 | — | — | | — | 1 |
| Drug 10 | 0.1 | — | — | — | — |
| Drug 14 | — | 0.1 | 0.1 | — | — |
| Drug 15 | — | — | — | 0.3 | — |
| Salt of a | 0.24 | 0.50 | 0.50 | 3.00 | 2.00 |
| Dialkyl | | | | | |
| Sulfo- | | | | | |
| succinate | | | | | |
| Second | 0.16 | 3.00 | 2.00 | 2.00 | 5.00 |
| Solvent 1 | | | | | |
| Penetration | — | 2.00 | — | — | — |
| Enhancer 4 | | | | | |
| Penetration | — | — | 3.00 | 5.00 | — |
| Enhancer 5 | | | | | |
| Emmollient | 8.00 | — | — | — | — |
| Total | 100.00 | 100.00 | 100.00 | 100.00 | 100.00 |

Drug 14 is trans-retinoic acid.

Drug 15 is adapalene.

Penetration Enhancer 4 is propylene glycol.

Penetration Enhancer 5 is dipropylene glycol.

TABLE 7

| | Example | | |
|---|---|---|---|
| | 33 | 34 | 35 |
| Mixture 1 | 86.5 | 93.9 | — |
| Mixture 8 | — | — | 96.73 |
| Drug 3 | 1 | 1 | 0.7 |
| Drug 5 | 2.5 | — | — |
| Drug 14 | — | 0.1 | 0.07 |
| Salt of Dialkyl | 4 | 2 | 1 |
| Sulfosuccinate | | | |
| Second Solvent 1 | 6 | 3 | 1.5 |
| Total | 100.00 | 100.00 | 100.00 |

Mixture 8 is a silicone adhesive in heptane or ethyl acetate, commercially available from Dow Corning Corporation under the trade name of Dow Corning® 7-4202 Silicone Adhesive. The silicone adhesive is present in an amount of 60 parts by weight, and the heptane or ethyl acetate is present in an amount of 40 parts by weight, each based on 100 parts by weight of the mixture.

All of the examples above exhibited excellent stability. In other words, no settling or separation is visually observed. In addition, no crystals or agglomerations are detected upon inspection of the compositions under microscope under 100× magnification. For purposes of the present invention, it is to be appreciated that various amounts of the mixtures may be used, other than those amounts shown above, such as quantities sufficient (qs) to achieve a desirable property such as viscosity and/or dosing of the drug. It is also to be appreciated that in the examples employing silicone adhesives, e.g. the examples of Table 5, the first solvent of the respective mixture is generally volatized off before applying the respective example to skin or performing any microbiological testing employing the example. As described above, when employing the silicone adhesive as the mixture, the first solvent serves primarily as a processing aid.

A zone of inhibition test protocol is performed in duplicate using a modified ASTM G21 method, on the composition of the present invention prepared in Example 16 and two comparison compositions, specifically Comparative Examples 1 and 2, to determine the effect of Drug 3, i.e., clindamycin phosphate, on the properties of the composition of Example 16 using P. acnes as a representative organism.

TABLE 8

| | | Comparative | Comparative |
|---|---|---|---|
| Zone of Inhibition Test | Example 36 | Example 1 | Example 2 |
| Mixture 1 | 93.00 | 96.00 | 98.00 |
| Drug 3 | 2.00 | 2.00 | 2.00 |
| Salt of a Dialkyl | 2.00 | — | — |
| Second Solvent 1 | 3.00 | — | — |
| Surfactant | — | 2.00 | — |
| Total | 100.00 | 100.00 | 100.00 |

Surfactant is a nonionic surfactant, specifically polysorbate 80, commercially available from Spectrum Chemical Mfg. Corp.

The zone of inhibition test protocol indicates that the composition of Example 36 is effective against P. acnes. The compositions of Comparative Examples 1 and 2 do not exhibit zones of inhibition. This test shows that the solubilizing effect of DOSS in the composition of Example 16 enables release of clindamycin phosphate.

The examples above show that drugs such as hydrophilic and lipophilic pharmaceutical agents are soluble and compatible in the compositions of the present invention. It is believed that this is due to the synergistic solubilizing property of DOSS, especially when dissolved in an alcohol, such as ethanol. The synergistic solubilizing property is able to compatibilize hydrophilic drugs in lipophilic materials such as the mixtures of the silicones and the first solvents.

The amounts of the salt of a dialkyl sulfosuccinate, e.g. DOSS, and the second solvent, e.g. ethanol, can be adjusted to maintain near saturation of the drug for optimum release of drugs. The activity of the drug does not change over time as demonstrated by the zone of inhibition test protocol.

Further, drugs with minimal solubility such as dapsone, are also soluble and compatible in the compositions. Hence drugs with a wide solubility range can be formulated in the compositions of the present invention. The compositions do not have a deleterious medium that can cause instability of the drug, and no water, pH change, or heat are required for preparing the compositions of the present invention. The compositions described herein also enhance chemical stability of the drugs via increased viscosities, such as when in film form, such as with the use of a PSA, or during storage, such as with the use of a silicone elastomer based mixture, e.g. a cyclomethicone and dimethicone crosspolymer. Since the drug is solubilized (or encapsulated) via the salt of a dialkyl sulfosuccinate, e.g. DOSS, is generally in a high viscosity medium, and is substantially free of water, the compositions of the present invention have enhanced chemical stability with regard to the drug.

The invention has been described in an illustrative manner, and it is to be understood that the terminology which has been used is intended to be in the nature of words of description rather than of limitation. Many modifications and variations of the present invention are possible in light of the above teachings, and the invention may be practiced otherwise than as specifically described.

1. A composition comprising:
a mixture of a silicone and a first solvent;
a drug;
a salt of a dialkyl sulfosuccinate for solubilizing said drug; and
a second solvent different from said first solvent.

2. A composition as set forth in claim 1 wherein said silicone is a silicone elastomer.

3. A composition as set forth in claim 2 wherein said first solvent comprises a polysiloxane for swelling said silicone elastomer.

4. A composition as set forth in claim 3 wherein said polysiloxane is selected from the group of hexamethyldisiloxane, hexamethylcyclotrisiloxane, hexadecamethylheptasiloxane, octamethyltrisiloxane, octamethylcyclotetrasiloxane, decamethyltetrasiloxane, decamethylcyclopentasiloxane, dodecamethylpentasiloxane, dodecamethylcyclohexasiloxane, tetradecamethylhexasiloxane, and combinations thereof.

5. A composition as set forth in claim 3 wherein said polysiloxane and said silicone elastomer are present in a weight ratio of 5:1 to 9:1.

6. A composition as set forth in claim 2 wherein said silicone elastomer is present in an amount of 5 to 20, said first solvent is present in an amount of 45 to 90, said drug is present in an amount of 0.0001 to 15, said salt of a dialkyl sulfosuccinate is present in an amount of 0.1 to 5, and said second solvent is present in an amount of 0.1 to 20, parts by weight, all based on 100 parts by weight of said composition.

7. A composition as set forth in claim 1 wherein said salt of a dialkyl sulfosuccinate comprises dioctyl sodium sulfosuccinate.

8. A composition as set forth in claim 1 wherein said second solvent comprises an alcohol.

9. A composition as set forth in claim 8 wherein said alcohol is a monomeric alcohol.

10. A composition as set forth in claim 9 wherein said monomeric alcohol is selected from the group of methanol, ethanol, propanol, butanol, pentanol, hexanol, heptanol, octanol, nonanol, decanol, and combinations thereof.

11. A composition as set forth in claim 8 wherein said salt of a dialkyl sulfosuccinate and said alcohol are present in a weight ratio of 1:1 to 1:4.

12. A composition as set forth in claim 1 wherein said drug is selected from the group of tetracycline, clindamycin phosphate, erythromycin, ciprofloxacin, norfloxacin, salicylic acid, benzoyl peroxide, alprostadil, terbinafine, clobetasol propionate, ketoconazole, progesterone, niacinamide, calcipotriol, retinoids, vitamin D analogues, vitamin A esters, dapsone, doxycycline, and combinations thereof.

13. A composition as set forth in claim 2 wherein said silicone elastomer comprises the reaction product of;
an ≡Si—H containing polysiloxane, and
an alpha,omega-diene,
in the presence of a metal catalyst.

14. A composition as set forth in claim 13 wherein said silicone elastomer is present in an amount of 75 to 95, said first solvent is present in an amount of 1 to 50, said drug is present in an amount of 0.0001 to 15, said salt of a dialkyl sulfosuccinate is present in an amount of 0.1 to 5, and said second solvent is present in an amount of 0.1 to 20, parts by weight, all based on 100 parts by weight of said composition.

15. A composition as set forth in claim 1 wherein said mixture comprises a cyclomethicone and dimethicone crosspolymer.

16. A composition as set forth in claim 1 wherein said mixture comprises a dimethicone and dimethicone crosspolymer.

17. A composition as set forth in claim 1 wherein said silicone is a silicone adhesive comprising the reaction product of;
a polydialkylsiloxane end blocked with silanols, and
a silicate resin.

18. A composition as set forth in claim 17 wherein said polydialkylsiloxane is a polydimethylsiloxane.

19. A composition as set forth in claim 17 wherein said first solvent is selected from the group of alkanes, arenes, esters, and combinations thereof.

20. A composition as set forth in claim 17 wherein said silicone adhesive is present in an amount of 40 to 80, said first solvent is present in an amount of 1 to 40, said drug is present in an amount of 0.0001 to 15, said salt of a dialkyl sulfosuccinate is present in an amount of 0.1 to 5, and said second solvent is present in an amount of 0.1 to 20, parts by weight, based on 100 parts by weight of said composition.

21. A composition as set forth in claim 1 further comprising an additive selected from the group of an emollient, a penetration enhancer, a fragrance, a drug solubilizer, a surfactant, an antioxidant, an antioxidant, a UV light blocker, a steroid, a pH adjuster, and combinations thereof.

22. A method of delivering a drug to a substrate, said method comprising the step of applying the composition of claim 1 to the substrate.

23. A composition comprising:
a silicone elastomer comprising the reaction product of;
an ≡Si—H containing polysiloxane having the formula
R₃SiO(R′₂SiO)ₐ(R″HSiO)_{b}SiR₃
wherein R, R′, and R″ are each independently alkyl groups with 1 to 6 carbon atoms, a is 0 to 250, and b is 1 to 250, and
an alpha,omega-diene having the formula
CH₂═CH(CH₂)ₓCH═CH₂
wherein x is 1 to 20,
in the presence of a metal catalyst and a polysiloxane,
a drug;
dioctyl sodium sulfosuccinate for solubilizing said drug; and
an alcohol for solubilizing said dioctyl sodium sulfosuccinate.

24. A composition as set forth in claim 23 wherein said polysiloxane is selected from the group of hexamethyldisiloxane, hexamethylcyclotrisiloxane, hexadecamethylheptasiloxane, octamethyltrisiloxane, octamethylcyclotetrasiloxane, decamethyltetrasiloxane, decamethylcyclopentasiloxane, dodecamethylpentasiloxane, dodecamethylcyclohexasiloxane, tetradecamethylhexasiloxane, and combinations thereof.

25. A composition comprising:
a silicone adhesive comprising the reaction product of;
a polydimethylsiloxane end blocked with silanols, and
a silicate resin,
a first solvent;
a drug; and
dioctyl sodium sulfosuccinate for solubilizing said drug.

26. A composition as set forth in claim 25 further comprising an alcohol different from said first solvent for solubilizing said dioctyl sodium sulfosuccinate.
